(19) United States
(12) Plant Patent Application Publication
(10) Pub. No.: US 2018/0035585 P1
(43) Pub. Date: Feb. 1, 2018

(54) Apple Tree Named 'STARAPPLE1'

(50) ;

(71) Applicant: STARGROW CULTIVAR DEVELOPMENT (PROPRIETARY) LIMITED, Stellenbosch (ZA)

(72) Inventor: Jeanne Fourie, Stellenbosch (ZA)

(21) Appl. No.: 15/330,026

(22) Filed: Jul. 27, 2016

(51) Int. Cl. A01H 5/08

(52) U.S. Cl. PLT/161

(57) ABSTRACT

‘Starapple1’ is new apple tree variety that in combination can be distinguished from other apple varieties by its percentage and intensity of red over-color; intense flavor with high sugar:acid ratio and balanced eating quality; red “bleeding” into the flesh at harvest; and scab resistance.

Genus and species: The apple tree variety of this invention is botanically identified as Malus domestica Mills.

Variety denomination: The variety denomination is ‘Starapple1’.

### BACKGROUND OF THE INVENTION

The invention refers to a new and distinct plant variety of apple tree (Malus domestica) named ‘Starapple1’. The new Malus variety is product of a planned and controlled breeding program for new Malus varieties. The objective of the breeding program is to select new Malus varieties with improved characteristics in color development, eating quality, production and disease resistance.

The new Malus variety was obtained through a controlled open pollinated cross. Seedlings of the population were top grafted on existing rootstocks. The first fruit of ‘Starapple1’ was observed in Feb 2005 from the top-grafted tree. Buds where selected from the top-grafted trees and 5 trees were propagated on M793 rootstock as second generation trees. Fruit form the second generation trees was first observed in 2008. ‘Starapple1’ was selected in 2009 after the second fruit crop form the second generation trees for further development because of the unique fruit quality traits observed over the two seasons consecutively.

### BRIEF SUMMARY OF THE INVENTION

The following traits have been repeatedly observed and are determined to be unique characteristics of ‘Starapple1’ which in combination distinguish this apple tree as a new and distinct variety:

1. Percentage and intensity of red over-color;

2. Intense flavor with high sugar:acid ratio and balanced eating quality;

3. Red “bleeding” into the flesh at harvest; and

4. Scab resistance.

The fruit of ‘Starapple1’ ripens between ‘Gala’ and ‘Golden Delicious’. Compared to ‘Gala’, fruit of ‘Starapple1’ develops a darker red over-color and has a significant different flavor with a higher sugar:acid ratio. Compared to ‘Golden Delicious’ fruit of ‘Starapple1’ ripens one week earlier and has a nearly full dark red over color where ‘Golden Delicious’ fruit has a greenish-yellow over color at harvest.

Fruit of ‘Starapple1’ develops a unique high intensity red over-color at harvest with some strippiness on the shaded side of the fruit. Fruit of ‘Starapple1’ develops a unique color intensity compared to other apple varieties ripening during the same period. When ripe some fruit shows strong red “bleeding” into the flesh from the skin, giving the ‘Starapple1’ a distinguishable flesh appearance.

### BRIEF DESCRIPTION OF THE PHOTOGRAPHS

The accompanying photographs illustrate the appearance of the new apple tree in production, fruit and internal quality of the fruit. The color of the photographs might vary slightly compared to the variety description, however the botanical description are the most accurately description of the true color of this new variety.

FIG. 1. Photograph showing a tree of this new variety, typically in production in an orchard.

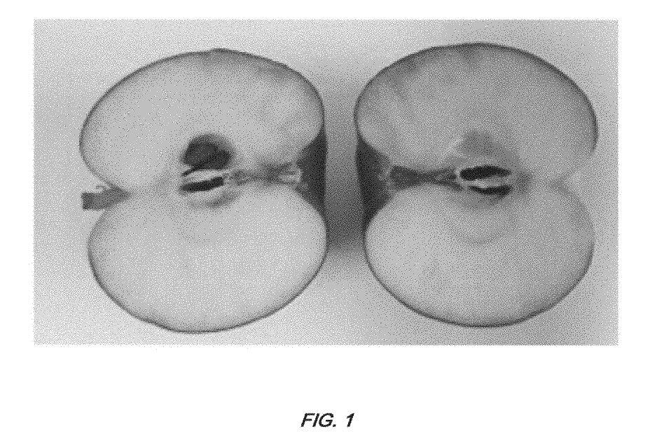

FIG. 2. Photograph of a fully developed fruit, close-up, on a tree grown in an orchard, showing the color development and shape of this new variety.

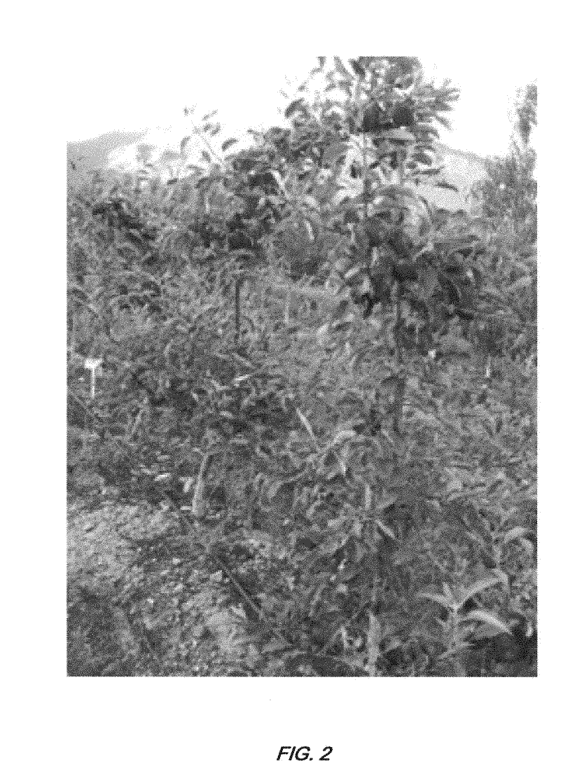

FIG. 3. Photograph of a central axis cut of fruit showing the white-cream flesh color with contrasting bleeding into the flesh close to the skin.

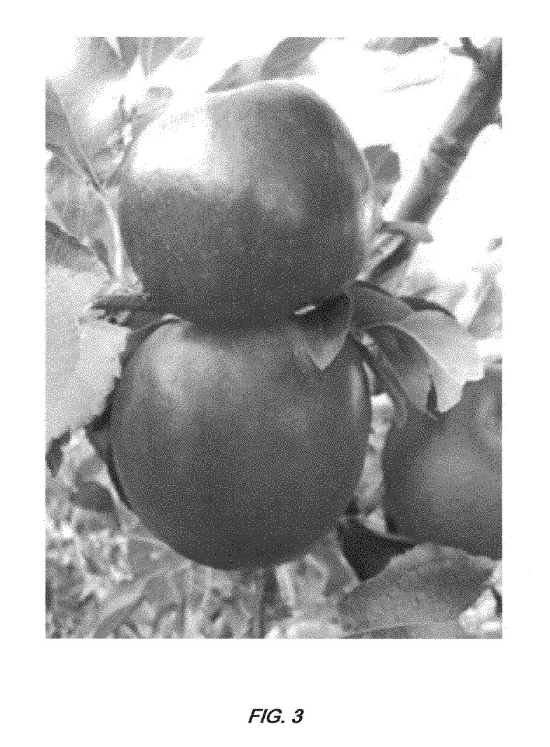

### DETAILED BOTANICAL DESCRIPTION

The photographs and botanical description are taken from trees grown at the Suikerbosrand Farm situated in the Koue-Bokkeveld near Ceres in the Western Cape, South Africa. The farm is situated in a main apple growing area and general commercial orchard practises is applied to the trees. The climate is temperate continental with low temperatures during winter and high temperatures during summer. The soil type is sandy rock with a micro-jet irrigation system. The photographs and description are believed to apply to plants of the variety grown elsewhere under similar soil and climate conditions.

The detailed botanical description is taken from over two fruiting season of trees budded on a standard apple rootstock. The data were collected and is reported according to official UPOV Variety Description requirements. The trees were five years old when the first observations were made. Measurements and numerical values represents averages for typical plants and plant parts. The actual measurements of any individual plant of plant parts, or any group of plants of plant parts, may vary from the stated average.
Plant description:
   Plant and growth habit.—Spreading, with strong growth habit. Bearing on both spurs and long shoots (2).
   Tree type.—Ramified (2).
   One-year-old shoot.—Shoot thickness: Medium (5). Length of internodes: Long (7). Color on sunny side: Dark Brown (5). Pubescence: Present, medium (5). Number of lenticels: Present, medium (5).
   Leaf description.—Leaf attitude: Outwards in relation to shoot (2). Length: Medium length (5). Width: Narrow width (3). Length width ratio: Large (7). Color: Medium green color intensity (5). Incision of margin: Biserrate (5). Pubescence: Present, Medium on the lower side (5).
   Petiole extent of anthocyanin coloration from the base.—Small-medium (4).
   Petiole length.—Medium (5).
Flower description:
   Flower type and flowering habit.—Small flower with medium pink color when unopened.
   Natural flowering season — beginning of flowering.—Medium (5).
   Petals.—Intermediate arrangement of petals (2).
Fruit description:
   Ripening time.—Typically early (3). End February, in SA, after Gala.
   Yield.—Moderate.
   Use.—Fresh.
   Length/height.—Long (7).
   Diameter.—Medium-Large (6). ±172 mm.
   Fruit weight.—±170 g, Medium (5). Ovoid shape (3).
   Depth of eye basin.—Medium.
   Width of eye basin.—Narrow.
   Fruit stalk diameter.—Medium (5).
   Fruit stalk color.—Green, with medium length (5), medium thickness (5) and deep stalk cavity (7).
   Fruit skin color.—Yellow ground color (3). Large relative over color (7). Red Hue (3). Medium intensity (5). Solid flush with weak stripes (2) and medium width of stripes (5). Absent or small area of fruit russet around stalk attachment, on cheeks or around eye basin (1).
   Lenticels.—Not prominent. Medium number (5). Medium size of lenticels (5).
   Flesh texture.—Crisp, firm.
   Flesh color.—Cream pinkish (2-5).
   Locules.—Fully open in cross section aperture (3).
   Seeds.—Ranging from 2 to 8 per fruit depending on environmental conditions. 8 mm length, 4 mm width. Brown color.

What is claimed is:

1. A new and distinct variety of apple tree having the characteristics substantially as described and illustrated herein.
